(19) United States
(12) Patent Application Publication
(10) Pub. No.: US 2008/0199962 A1
(43) Pub. Date: Aug. 21, 2008

(54) Qualitative Assay of Extra-Virgin Olive Oils

(76) Inventors: Ferdinando Ippolito, Roma (IT); Antonia Macciouccu, Roma (IT); Siro Passi, Roma (IT); Aldo Di Carlo, Roma (IT)

Correspondence Address: YOUNG & THOMPSON, 209 Madison Street, Suite 500, ALEXANDRIA, VA, 22314, US

(21) Appl. No.: 12/063,261

(22) PCT Filed: Aug. 7, 2006
(86) PCT No.: PCT/IT06/00608

§ 371 Date: Feb. 8, 2008

(30) Foreign Application Priority Data
Aug. 8, 2005 (IT) RM2005A000435

(51) Int. Cl. G01N 33/03

(52) U.S. Cl. 436/60

(57) ABSTRACT

A methodology that enables introduction of an innovative assay for evaluating the extra-virgin olive oil (EOO), based upon the quantification of the main components of the saponifiable and non-saponifiable fractions of EOOs and upon its total antioxidizing power.

The present invention relates to the sector of merceological evaluation of olive oils and more in particular relates to a method for evaluating olive oil based upon a plurality of chemico-physical indices, which enables classification of the oils not according to an organoleptic examination that judges the oil from the standpoint of its visual and olfactive characteristics and characteristics of taste, nor on the basis of the percentage of fatty matter alone and its degree of acidity, but rather according to its contents of non-saponifiable molecules such as phenols and polyphenols including vitamin Er phytosterols, squalene, aromatic compounds, etc., the high levels of which determine the intrinsic quality of an extra-virgin oil. This depends upon a complex interaction of factors that regard the cultivar, the degree of ripening of the olives, the season and year of the harvest and the climate, as likewise the period and the method of warehousing of the olives and of the oil.

Up to the present day, an instrument based upon chemico-physical parameters capable of evaluating the quality of an olive oil was not available. The quality recognized for extra-virgin oils is considered currently the resultant of two different types of investigation: on the one hand, the chemico-physical analysis, which is limited to ascertaining the actual percentage composition of the fatty matter and its degree of acidity; on the other hand, the organoleptic examination, which judges the oil from the standpoint of its visual and olfactive characteristics and its characteristics of taste and evaluates the qualities and defects thereof. It is important to point out that olive oil is the first foodstuff product for which sensorial analysis, based upon the panel-test system (a particular standardized analytical methodology in which committees made up of expert tasters, selected from among the ones enrolled in a national list, operate) constitutes a merceological discriminant.

In the legal classification of olive oils, Italy has conformed to the Regulations of the European Community, so that the extra-virgin oil currently consumed as foodstuff recognizes as qualitative parameter a percentage content of free oleic acid not higher than 1 g per 100 g of product. Extra-virgin olive oil (EOO) is distinguished from the other vegetable oils in so far as it is produced on a non-industrial scale using exclusively mechanical systems, without further refinements, which would reduce substantially the uncommon organoleptic properties of EOO. Instead, it is known that seed oils are obtained either by applying pressure followed by extraction using hexane or via extraction using organic solvents. The crude oils thus obtained are then subjected to processes of purification from rubbers and polymers, de-acidification, whitening and deodorization.

The finding that in the Mediterranean area there is a reduced incidence of cardiovascular diseases has led to the hypothesis that a diet rich in cereals, legumes, fresh fruit and vegetable, fish, wine in moderate amounts and EOO can exert beneficial effects on human health. These effects have been principally attributed to the low level of saturated fatty acids, to a high presence of mono-unsaturated fatty acids, and to the optimal ratio between the polyunsaturated fatty acids (PUFAs) of the n-6 and n-3 series present in EOO and in the Mediterranean diet.

In fact triglycerides, which are the most abundant lipidic fraction in EOO, contain a high percentage of oleic acid (C18:3, 65-78%), an appreciable amount of linoleic acid (C18:2 n-6, 6-9%) and alpha-linolenic acid (C18:3 n-3, 0.4-0.8%), and relatively low levels of saturated fatty acids, such as palmitic acid (12-18%) and stearic acid (1.5-2.5%). Oleic acid is much more resistant than PUFAs to lipoperoxidative processes and is, to some extent, responsible both for the plasmatic increase of HDL-C (high-density lipoproteins-cholesterol) and apoprotein AI and for the plasmatic decrease of LDL-C (low-density lipoproteins-cholesterol) and apoprotein B. It is, in other words, involved positively in the prevention of cardiovascular diseases (atherosclerosis, myocardial infarction, cerebral stroke, etc.), which represent the main cause of mortality in industrialized countries.

In any case, re-valorization of EOO as best vegetable oil in absolute terms for humans from the clinico-dietetic standpoint (its consumption is in considerable increase even in countries that do not produce it, such as USA, Canada and Japan) cannot be due exclusively to its composition in fatty acids, since there exist many other oils, for example hazelnut oil or macadamia oil, which, albeit containing a composition in fatty acids very similar to that of EOO, are not equally beneficial for human health. In addition to triglycerides and to the free fatty acids, EOO contains in fact a large number of non-saponifiable molecules, such as phenols and polyphenols including vitamin E, phytosterols, squalene, aromatic compounds, etc., which reach a concentration of 1-2% and differentiate it from the other oils.

Antioxidants, squalene and sterols form part of the non-saponifiable fraction of EOO, i.e., of that complex fraction which, since it does not contain free or esterified fatty acids, does not become soap if treated with alkalis. It is likely that a large part of the dietetic, therapeutic and cosmetic properties of EOO are to be attributed precisely to the non-saponifiable fraction.

The present applicants have highlighted that oxidation of EOO in the presence of air (self-oxidation) has as its main “target” not so much polyunsaturated fatty acids, but rather the antioxidants of the non-saponifiable fraction and in particular vitamin E, ortho-diphenols and squalene, which tend towards oxidative degradation. In a way similar to what occurs in many biological systems subjected in vivo or in vitro to oxidative stress, an evident picture emerges, in which the various antioxidants do not operate autonomously, but collaborate actively and synergistically to prevent damage to the polyunsaturated acids. Vitamin E is the most important, representative and functional lipophilic antioxidant present in EOO and is the first to be oxidized, becoming, in part, pro-oxidant, and consequently able to oxidize the other antioxidants of the non-saponifiable fraction, in particular ortho-diphenols and squalene, with formation of other reactive species, of a radical nature and otherwise. There is consequently triggered a perverse self-stimulating radical mechanism, which, in the end, with the massive reduction of antioxidants, will regard the polyunsaturated acids of the oil. In the course of said oxidative process, innumerable molecules are generated, carbonylic and otherwise, more or less presenting characteristics of toxicity and mutagenicity, and such as to impart, even at extremely small concentrations, an extremely unpleasant smell and taste on the EOO (rancidity).

As has already been mentioned previously, the level of the non-saponifiable fraction depends upon a complex interaction of factors, which regard the cultivar, the degree of maturation of the olives, the season of the harvest and the climate, the soil, the period, the lot and year, the method of warehousing of the olives and of the oil, etc.

In particular, phenols play an important role:

in the stability and conservation of EOO; and

in the flavour and unique taste of EOO; some phenols are able to bestow the characteristic bitterish taste (phenolic acids) and/or spiced taste (secoiridoids), which, in some cases, recall the taste of fruit or a peppered taste. In general, from greener olives a “fruity” olive oil is produced, which is richer in phenols than “sweet” oil, which derives from ripe olives with low levels of phenols.

In addition, phenols represent in the diet biologically active compounds, which are correlated with a low risk of development of cardiovascular diseases. In particular d-RRR-α-tocopherol (vitamin E) and o-diphenols (hydroxytyrosol, hydroxytyrosol-aglycones, oleuropein-aglycones, etc.) contribute to the stability of EOO and to its antioxidizing properties and its properties as scavenger of radicals, which enable prevention of oxidation of lipoproteins, which are primarily involved in development of arteriosclerosis.

Another compound that should be highlighted is squalene (0.4-0.9%), the molecule present in highest concentration in the non-saponifiable fraction. Squalene is a triterpenoid hydrocarbon with thirty carbon atoms and with six double bonds, which represents the precursor of the synthesis of cholesterol in humans and of beta-sitosterol (and other phytosterols) in EOO. It is found also in the liver of sharks (whence the name) and in human sebum, where its level reduces with age and is able to contrast the reactive species of oxygen and other free radicals induced by UV irradiation of the skin, in this way behaving as indirect natural UV filter. This provides some, albeit tardy, explanation of the reason for the traditional cosmetic use of EOO by our ancestors, above all in the South of Italy, in Greece and in the Mediterranean countries in general.

As has been said, the concentration of squalene in olive oil is higher than that of phenols so that the presence of very low levels can constitute a significant “marker” in a possible process of rectification undergone by olive oil.

On the basis of said recent acquisitions, deriving from studies conducted in the dermatological field and in particular on seborrhoic dermatitis at IRCCS (Istituto dermatologic San Gallicano), and IDI of Rome (Centro studi sull'invecchiamento cellulare—Centre for Studies on Cellular Ageing) by Prof. Ferdinando Ippolito and by Prof. Siro Passi, respectively, a methodology has been developed that enables introduction of an innovative method for evaluating EOO, based upon quantification of the main components of the saponifiable and non-saponifiable fractions of EOO and upon its total antioxidizing power.

The innovation, which involves transcription of the analytical data in the form of a sort of report on the product on sale to the public, is a document that validates the quality of EOO, justifying the adequacy of its price.

According to the present invention, the valid parameters for the purposes of identification of the biochemical quality of the oil are the following:

TABLE I
Fatty acids in % of the triglyceride fraction

C14:0
C16:0
C17:0
C17:1 n-7
C18:0
C18:1 n-9
C18:2 n-6
C18:3 n-6
C20:0
C20:1
C22:0
C24:0
Acidity number (% of oleic acid)
Number of peroxides (mEq. O₂/kg)
Total sterols (mg/kg)
Campesterol (%)
Stigmasterol (%)
β-sitosterol (%)
Δ-5-avenasterol (%)
Cholesterol
Others (%)
Total antioxidizing power (mmol
Trolox/kg)
(d-RRR-α-tocopherol) (mg/kg)
d-RRR-γ-tocopherol (mg/kg)
β-carotene (mg/kg)
Squalene (mg/kg)
Total phenols (mg/kg) (Folin-
Chocaltau)
Tyrosol (mg/kg)
Hydroxytyrosol (mg/kg)

All these parameters concur in defining the high quality of an extra-virgin olive oil. Of particular interest is the total antioxidizing power, which represents the sum of the antioxidizing activities of the various molecules present in the non-saponifiable fraction. A slight variation in time (<10%) thereof with respect to the moment of production of EOO indicates an optimal conservation of said oil. On the other hand, a significant reduction thereof is an indication of a serious degrading process that will lead inevitably to rancidity, an irreversible oxidative process that will be all the more rapid, the higher the reduction of the total antioxidizing power.

In support of what has been said, provided hereinafter are the experimental results regarding a study on the stability of different oils over time.

### 1. Stability Over Time of the Active Principles Of the Saponifiable and Non-Saponifiable Fractions of EOO

Foreword

Forty-two (42) extra-virgin oils were studied, of which forty-one (41) were Italian oils coming from various regions and one (1) came from the United States (N° 37), in addition to four (4) Spanish oils, which were not extra-virgin, but were rectified and to which vitamin E and other molecules were added. Two sealed (topped) bottles of each oil were purchased regularly at retail outlets, at variable prices ranging from 4.25 ε/l (S. Sabina classico—Lazio, Farchioni—Umbria) to 28.26 ε/1 (Castello Banfi—Tuscany) or directly from the oil-presses (3.5-5 ε/1). Each bottle was opened, and 20-25 ml of oil were sampled for the analysis (T=0, a). The bottles were then re-closed hermetically and stored in the dark at room temperature. The amount sampled had thus been replaced with air.

The same analyses were conducted after one year (T1=1 year, b), two years (T2=2 years, c) and three years (T3=3 years, d) on the same bottles. After three years also the remaining non-used bottles were opened, which up to then had remained sealed, and the respective oils were analysed (T3b=3 years, e).

Acidity

Acidity is the main factor, but not the only one, that characterizes the extra-virginity of an oil (acidity in oleic acid <1%). The only oil, apart from the Spanish oils, that did not correspond to the requisites of extra-virginity was the Californian one (California Harvest EV, N° 37), which had an acidity of 1.19 and consequently was to be considered as virgin olive oil.

After one year, two years, and three years, the acidity increased on average by approximately 5% also in the sealed bottles.

Fatty Acids

Fatty acids in the oils are present above all in the triglyceride fraction, which represents the almost entirity of the oils (≈97%). It appears evident that the pattern of fatty acids of the various oils fell within the limits fixed by law, with minimal occasional exceptions, which, in any case, do not constitute a proof of fraud (C16:0: 10.5-17.4%; C16:1: 0.4-2.1%; C17:0: 0.04-0.2%; C17:1: 0.09-0.4%; C18:0: 2.1-4.1%; C18:1: 69.4-76.2%; C18:2: 4.5-9.8%; C18:3 n-3: 0.4-1.1%; C20:0: 0.3-0.8%; C20:1: 0.2-0.5%). On the other hand, the quality of an EOO cannot be based upon on its high percentage of mono-unsaturated oleic acid and its low levels of polyunsaturated acids (C18:2 n-6+C18:3 n-3), since there exist various other vegetable oils, in particular hazelnut oil and macadamia oil, which have the same pattern of fatty acids, but are not comparable, from the alimentary and therapeutic standpoint, to EOO. The main quality of EOO lies in its non-saponifiable fraction.

After one, two and three years, no significant variations of fatty acids were observed.

UV Spectrophotometric Analysis

According to the official method envisaged by the Regulations of the European Community, the determination of the specific-extinction coefficients at 232 and 270 nm and the corresponding ΔK for the extra-virgin olive oils envisages the following values: K232<2.40; K270<0.20; ΔK<0.01. Altered values were found not only in the four Spanish oils, which were declaredly rectified, but also in the oil N° 26 (S. Cristina, Lombardy; 21.18 ε/l), which showed a high K232 and in the oil N° 29 (Castello Banfi, Tuscany; 28.26 ε/1), which showed high K232 and K270.

After one, two and three years the constants had increased systematically by approximately 5% a year, whilst they had increased by approximately 10% in the ones sealed and opened only after three years.

Lipoperoxides

According to the official method envisaged by the Regulations of the European Community the level of lipoperoxides in EOO must not exceed 20 mEq. O₂/kg. The oils under study did not exceed said limit even though over the three years of study the value of lipoperoxides increased systematically by 5-10% a year in the bottles previously opened and then re-closed (and this is certainly due to the increase in air present therein), whereas it increased by approximately 15% in the ones sealed and opened only after three years.

β-Carotene

The official method envisaged by the Regulations of the European Community does not fix the levels in EOO of β-carotene, which is a powerful lipophilic antioxidant precursor of vitamin A. The concentrations of said carotenoid in the oils under examination were markedly variable and not very high (0.50-3.24 μg/g at the start). The levels of β-carotene in the oils are in strict correlation with those of chlorophyll. Chlorophyll, in fact, under the action of UV radiation, generates singlet oxygen, a very toxic reactive species of oxygen, which is readily neutralized precisely by β-carotene. This indicates that, the greener the olives, the richer the respective oils are in chlorophyll and β-carotene. When the olives are completely ripe (black), the levels of β-carotene and chlorophyll in the oils are extremely low if not zero.

After one, two and three years the levels of β-carotene reduced by approximately 10-15% a year in the bottles previously opened and then re-closed, whilst they reduced by approximately 20% in the ones sealed and opened only after three years.

Squalene

Squalene, which is a precursor of the synthesis of sterols, is the molecule present in the highest concentration in the non-saponifiable fraction of EOO. Also for this triterpenoid hydrocarbon with thirty carbon atoms, the official method envisaged by the Regulations of the European Community does not fix the levels in EOO, which in any case are markedly variable (3.02-8.39 mg/g). In theory, they should be around 0.5-0.9% of the oil and their variability can be ascribed both to a (self)-oxidative process that reduces the concentration thereof, and to the degree of ripening of the olives before being pressed. In fact, the levels of squalene for one and the same cultivar increase with the ripeness of the olives. In other words, an oil with a greenish colour will present, at least initially, discrete values of chlorophyll and β-carotene and low values of squalene. Instead, an oil with an initial intense yellow colour will have high concentrations of squalene and low concentrations of β-carotene and chlorophyll.

After one, two and three years the levels of squalene reduced by 10-15% a year in the bottles previously opened and then re-closed, whilst they reduced by approximately 20% in the ones sealed and opened only after three years.

d-RRR-α-tocopherol (Vitamin E)

The official method envisaged by the Regulations of the European Community does not fix the levels in EOO of this essential antioxidant, d-α-tocopherol or d-RRR-α-tocopherol, which plays a primary role in the stability of the membrane and which, as in the case of β-carotene and squalene, was markedly variable in the oils under study (from 0.15 to 0.44 μg/ml). In any case, the oils from Calabria had a higher concentration of d-RRR-α-tocopherol as compared to the other oils and this, in part, could depend upon the fact that the olives in Calabria grow in terrains that are very rich in iron. It should be emphasized that 15-20 ml/day of oil from Calabria correspond to the RDA (recommended dietary allowance) for vitamin E. Together with d-RRR-α-tocopherol there was a presence of markedly variable levels, albeit low, of d-RRR-γ-tocopherol, (3.8-4.9 μg/ml), also this belonging to the group of vitamin E, but being much less effective and functional in vivo than α-tocopherol. It is important to emphasize that in all the oils studied, including the rectified Spanish ones, d-α-tocopherol is in actual fact d-RRR-α-tocopherol and not d,l-α-tocopherol (which is an index of sophistication), as was possible to verify by means of a chiral HPLC column.

After one, two and three years the levels of α-tocopherols and γ-tocopherols reduced by 10-15% a year in the bottles previously opened and then re-closed, whilst they reduced by approximately 15% in the ones sealed and opened only after three years.

Sterols

According to the official method envisaged by the Regulations of the European Community the level of total sterols in EOO should be ≈1 mg/g, the percentage of P-sitosterol should be ≈93% of all sterols, whilst that of campesterol should be ≦4%. Said limits, which apply also for the other types of oils (virgin olive oil, current virgin olive oil, lamp oil, refined oil, olive oil, etc.) did not appear as being in line with the standard for all the oils under examination. It should be recalled that the official Regulations maintain that “it is enough for one characteristic not to conform to the values indicated for the oil to change category or be deemed not to conform to the purity standard”. Certainly, it appears difficult to claim that the aforementioned oils should not be considered compliant only for some anomalous value of a sterol.

In any case, the percentage of the various sterols did not vary in the course of the three years of investigations.

Phenols

The official method envisaged by the Regulations of the European Community does not fix the levels of phenols in EOO, which in any case are markedly variable. For their determination a spectrophotometric method is in general employed, which uses the Folin-Chocalteau reagent, which is very unspecific in so far as it measures the total amount of phenols, without providing any detail as regards the individual molecules, and is subject to interference capable of altering the final result. In any case the range of values found was between 44 and 142 mg of gallic acid/kg.

Also the levels of simple monophenols and diphenols, identified unequivocally and quantified by GC-MS are markedly variable: cinnamic acid (0.25-1.17 μg/g), tyrosol (8.65-19.14 μg/g), 4-OH benzoic acid (0.12-0.90), vanillic acid (0.11-0.41), isovanillic acid (0.02-0.19 μg/g), hydroxy tyrosol (3.64-32.50 μg/g), protocatechuic acid (0.08-0.59 μg/g), syringic acid (0.02-0.22 μg/g), p-coumaric acid (0.12-0.37 μg/g), ferulic acid (0.05-0.45bμg/g), caffeic acid (0.02-0.12 μg/g), and chlorogenic acid (0.01-0.11 μg/g).

In the course of the three years of investigation, the levels of simple diphenols had reduced to a much larger extent as compared to that of monophenols: 30-40% vs. 10-15%.

On some oils the levels of complex (poly)-phenols were studied by HPLC-MS (FIG. 2). From the results obtained it may be appreciated that they represent the most unstable molecules of EOO (they degrade more rapidly as compared to the other aforementioned molecules, i.e., approximately 60% over the three years).

Total Antioxidizing Power (RAC Method and TEAC Method)

The measurement of the total antioxidizing capacity of the oils under study as compared to Trolox (6-OH-2.5.7.8-tetramethyl-2-carboxylic acid) was conducted with the RAC (reducing/antioxidant capacity) method, which uses chemiluminescence as detecting system and is based upon the capacity of an antioxidant for neutralizing the hydroxyl radicals generated by cytochrome C in the presence of hydrogen peroxide. The results obtained with this method (586-1878 μmol TE/kg) were compared with the ones obtained with the well-known TEAC (Trolox equivalent antioxidant capacity) method (836-1884 μmol TE/kg).

The results obtained after three years revealed a reduction of 25-30% of the total antioxidizing power in the bottles previously opened and then re-closed, whilst a reduction of approximately 15% was noted in the ones sealed and opened only after three years.

### Conclusions

From an analysis of the experimental results on the forty-five oils, it is possible to draw some obvious conclusions.
The extremely high disparity of prices seems decidedly excessive, unless one wishes to justify it on the basis of extremely subjective organoleptic evaluations that are, by their very nature, somewhat arbitrary. Said evaluations do not go beyond a classification of an EOO on the basis of a numeric scale established in relation to the perception of the characteristics of its flavour, according to the judgement of a group of selected tasters constituting a panel. In other words, a group of so-called “experts” establishes the organoleptic characteristics of an EOO and its cost, whereas biochemical analyses can unequivocally determine such parameters as well as many others that are certainly less sense-based and more reliable from the point of view of human health. If an oil is rancid, it is bitter or tastes of machine grease or dregs and certainly does not call for experts to establish it.
The stability of EOOs is an extremely serious problem. If already after just one year from their production, some requirements of quality concerning the oils are reduced significantly, it is necessary to resort to remedies. First of all, it is necessary to be sure that said requirements are met prior to bottling. It would be necessary then to study a kinetics of degradation, and finally to put an expiry date on the label.
Establishing once and for all that the quality in a broad sense, including the so much exalted flavour, of an EOO is not so much due to its pattern of fatty acids (the same pattern can occur with seed oils or with refined olive oils or sansa oils, etc.), but rather to the qualitative-quantitative composition of its non-saponifiable fraction. Consequently, the pattern of fatty acids and the composition of the main molecules of the non-saponifiable fraction should be indicated on the label, as a sort of report, with the indication of their expiry date or period of stability, as indicated in TABLE I.

The experiments conducted have enabled identification of the range of values within which the pattern of fatty acids and of the main molecules of the non-saponifiable fraction deriving from the analysis must fall in order to guarantee a sufficient stability to oxidative stress of the oil in question. The data appear in TABLE II below.

More in particular, it has been shown that, if the values of analysis fall within the range indicated below, the period of stability of the oils, if conserved in well-sealed bottles of a dark brown or dark green colour, is at least two years.

TABLE II

| Fatty acids in % of the | |
|---|---|
| triglyceride fraction | Range of values |
| C16:0 | 12.0-17.0% |
| C16:1 | 0.4-1.0% |
| C17:0 | 0.05-0.2% |
| C17:1 n-7 | 0.05-0.2% |
| C18:0 | 1.8-3.0% |
| C18:1 n-9 | 68.0-76% |
| C18:2 n-6 | 5.0-10.0% |
| C18:3 n-6 | 0.5-0.9% |
| C20:0 | 0.3-0.5% |
| C20:1 | 0.1-0.4% |
| C22:0 | 0.05-0.2% |
| C24:0 | 0.05-0.2% |
| Σ saturated acids | 15.0-19.0% |
| Σ mono-unsaturated acids | 71.0-77.5% |
| Σ polysaturated acids | 6.0-10.4% |
| Acidity number (% of oleic acid) | 0.2-0.7 |
| Number of peroxides (mEq. O₂/kg) | 6.0-15.0 |
| Total sterols (mg/kg) | 1050-1350 |
| Campesterol (%) | 2.7-3.9 |
| Stigmasterol (%) | 1.5-2.9 |
| β-sitosterol (%) + Δ-5- | 89.0-94.0 |
| avenasterol (%) | 0.1-0.3 |
| cholesterol (%) | 0.5-2.0 |
| others (%) | |
| Total antioxidizing power (mmol | 1000-2000 |
| Trolox Equiv./kg) | |
| TEAC method | 1000-2000 |
| RAC method | |
| d-RRR-α-tocopherol (mg/kg) | 200-400 |
| d-RRR-γ-tocopherol (mg/kg) | 3.0-10.0 |
| β-carotene (mg/kg) | 1.0-2.5 |
| Squalene (mg/kg) | 4000-8000 |
| Total phenols (Folin-Chocaltau, | 70-200 |
| mg gallic acid/kg) | |
| Tyrosol (mg/kg) | 7-20 |
| Hydroxytyrosol (mg/kg) | 10-25 |

As a confirmation of what has been set forth above, three EOOs were subjected to a series of tests of oxidative stress induced in various ways.

Three Italian EOOs of different origin were taken in consideration: Veroli (Province of Frosinone), Camino (Province of Viterbo) and Scalicelle (Province of Caserta), purchased in a common retail outlet.

All the oils were exposed to different conditions of oxidative stress:

a) heating to 100° C. up to 3 hours;

b) heating to 180° C. up to 30 minutes;

c) heating to 200° C. up to 30 minutes;

d) UV radiation up to 30 minutes;

e) heating in a microwave oven (m.w.) at a power increasing up to 600 W for 10 minutes;

f) self-oxidation in air up to 3 months

For each oil the following parameters were measured:
variations of the pattern of fatty acids;
acidity;
total antioxidizing capacity with respect to Trolox measured via two methods: RAC and TEAC;
lipoperoxides (DEPD=diethyl p-phenylenediamine sulphate);
levels of total phenols (Folin-Chocalteau);
levels of α-tocopherols and γ-tocopherols and squalene.

The results are given in Tables III and IV appearing below.

TABLE III

| | | | 100° C. | 180° C. | 200° C. | 200° C. | UV | air | m.w., |
|---|---|---|---|---|---|---|---|---|---|
| Oil | | t = 0 | 3 h | 30 min | 15 min | 30 min | 30 min | 90 g | 600 W × 10 min |
| Veroli | S | 16.9 | 17.6 | 17.8 | 17.8 | 18.4 | 18.2 | 17.5 | 17.7 |
| | M | 72.8 | 72.6 | 72.4 | 72.2 | 71.9 | 72.2 | 73.1 | 72.7 |
| | P | 10.3 | 9.8 | 9.8 | 10.0 | 9.7 | 9.6 | 9.4 | 9.6 |
| Canino | S | 17.3 | 18.6 | 18.4 | 17.9 | 18.8 | 18.9 | 17.9 | 18.1 |
| | M | 75.0 | 74.4 | 74.7 | 74.8 | 74.8 | 74.6 | 74.8 | 74.7 |
| | P | 7.7 | 7.0 | 6.9 | 7.3 | 6.8 | 6.7 | 7.1 | 7.2 |
| Scalicelle | S | 16.9 | 18.1 | 18.6 | 17.8 | 18.9 | 18.5 | 17.8 | 17.5 |
| | M | 71.3 | 71.5 | 70.2 | 71.3 | 70.8 | 70.8 | 71.0 | 71.3 |
| | P | 11.8 | 10.4 | 11.1 | 10.9 | 10.3 | 10.7 | 11.2 | 11.2 |

Appearing in the above Table III are the percentages of saturated fatty acids (S), mono-unsaturated fatty acids (M) and polyunsaturated fatty acids (P) in the different oils subjected to the various types of oxidative stress. Each result is the average on 5 determinations SD<7%.
p<0.001 and p<0.01 vs. t=0

Pattern of fatty acids—As may be seen from the table, the fatty acids of the three EOOs did not undergo any significant variations following upon the various oxidative stresses.

Appearing in following Table IV are the levels of α-tocopherols and γ-tocopherols, lipoperoxides (DEPED), total phenols (Folin-Chocalteau), acidity and total antioxidizing power (RAC method and TEAC method) of the three EOOs subjected to the same oxidative stresses.

TABLE IV

| | Acidity | RAC | TEAC | Folin-Chocalteau | DEPD | α-Toc. | γ-Toc. |
|---|---|---|---|---|---|---|---|
| | % Oleic ac. | μmolTE/Kg | μmolTE/Kg | mg gallic Acid/Kg | mEqO₂/Kg | mg/g | μg/g |
| t = 0 | 0.19 | 1589 | 1498 | 103.8 | 4.2 | 0.20 | 5.39 |
| 100° C. 1 h | 0.19 | 1528** | 1299* | 101.3 | 6.2* | 0.18 | 5.30 |
| 100° C. 2 h | 0.19 | 1518* | 1164* | 85.1* | 7.5* | 0.15** | 4.96* |
| 100° C. 3 h | 0.20 | 1508* | 1039* | 68.3* | 9.8* | 0.13* | 4.52* |
| 180° C. 15 min | 0.20 | 1500* | 1249* | 88.8* | 7.1* | 0.14* | 4.68* |
| 180° C. 30 min | 0.20 | 1117* | 1190* | 73.9* | 4.5* | 0.08* | 3.92* |
| 200° C. 15 min | 0.20 | 1401* | 1090* | 85.0* | 13.6* | 0.11* | 4.49* |
| 200° C. 30 min | 0.21 | 866* | 928* | 71.0* | 7.9* | 0.09* | 2.83* |
| Air 1 month | 0.20 | 1226* | 1212* | 96.0** | 8.6* | 0.15** | 5.12* |
| Air 2 months | 0.21 | 878* | 1164* | 85.3* | 18.3* | 0.13* | 4.78* |
| Air 3 months | 0.23 | 554* | 931* | 79.2* | 39.3* | 0.08* | 4.36* |
| UV 10 min | 0.20 | 1331* | 1307* | 93.2* | 10.6* | 0.15** | 4.13* |
| UV 15 min | 0.21 | 1176* | 1103* | 81.7* | 16.3* | 0.12* | 3.35* |
| UV 30 min | 0.21 | 87 4* | 877* | 74.2* | 33.5* | 0.09* | 2.86* |
| m.o. 100 W/10 min | 0.20 | 1338* | 1278* | 91.2* | 7.4* | 0.14* | 2.86* |
| m.o. 400 W/10 min | 0.21 | 450* | 892* | 51.2* | 4.8 | 0.08* | 1.52* |
| m.o. 600 W/10 min | 0.21 | 56* | 377* | 10.2* | 3.6 | 0.03* | 0.33* |

From the tabulated data the findings were as follows.

Acidity—The three EOOs showed the same behaviour: the acidity did not vary excessively following upon the various oxidative stresses, whilst there was a drastic reduction in the total antioxidizing capacity (RAC and TEAC methods), the levels of tocopherols (α>γ), and the levels of total phenols, and there was a significant increase in the levels of lipoperoxides (DEPD). The latter parameter deserves to be examined in greater depth. The highest and most rapidly increasing values were obtained with self-oxidation in air up to three months and with UV irradiation. Heating to 180° C. and 200° C. yielded higher values after 15 minutes than after 30 minutes. Of particular interest is heating in the microwave oven, which caused the greatest oxidative damage as compared to the other conditions, i.e., the maximum reduction in the total antioxidizing capacity and in the levels of tocopherols and total phenols. The level of lipoperoxides decreased, instead, even as compared to the values corresponding to t=0, with the increase in power. This is probably due to a termination reaction between lipid radicals and non-neoformed ones.

### Conclusions

In the three EOOs subjected to different conditions of oxidative stress the significant reduction in the total antioxidizing capacity (RAC and TEAC methods), in the levels of tocopherols (α>γ), and in total diphenols is associated to the significant increase in the levels of lipoperoxides (DEPD). The increase in lipoperoxidation does not lead to a reduction in the polyunsaturated acids, to which the increase in the number of peroxides is generally attributed. In EOOs oxidative stress, however triggered, does not seem to affect significantly the pattern of fatty acids.

1-5. (canceled)

6. A method for evaluating olive oil, characterized in that it envisages obtaining, for each lot of oil, by means of chemico-physical analyses, a plurality of chemico-physical parameters that measure, in addition to the content of triglycerides and free fatty acids in the oil analysed, the content of non-saponifiable molecules, such as phenols and polyphenols including vitamin E, phytosterols, squalene and aromatic compounds, the high level of which determines the intrinsic quality of an extra-virgin oil; said parameters concurring with one another to identifying the total antioxidant power of the oil itself.

7. The method according to claim 6, wherein the chemico-physical parameters to be analysed are the following:
Fatty acids in % of the triglyceride
fraction
C14:0
C16:0
C17:0
C17:1 n-7
C18:0
C18:1 n-9
C18:2 n-6
C18:3 n-6
C20:0
C20:1
C22:0
C24:0
Acidity number (% of oleic acid)
Number of peroxides (mEq. O₂/kg)
Total sterols (mg/kg)
Campesterol (%)
Stigmasterol (%)
β-sitosterol (%)
Δ-5-avenasterol (%)
Cholesterol
Others (%)
Total antioxidant power (mmol Trolox/kg)
(d-RRR-α-tocopherol) (mg/kg)
d-RRR-γ-tocopherol (mg/kg)
β-carotene (mg/kg)
Squalene (mg/kg)
Total phenols (mg/kg) (Folin-Chocaltau)
Tyrosol (mg/kg)
Hydroxytyrosol (mg/kg)
where the total antioxidant power is obtained by means of the RAC (reducing/antioxidant capacity) method, which uses chemiluminescence as detecting system and is based upon the capacity of an antioxidant for neutralizing the hydroxyl radicals generated by cytochrome C in the presence of hydrogen peroxide.

8. The method according to claim 7, characterized in that, in order to verify that the stability of the oil analysed, conserved in a sealed dark brown or dark green bottle, is at least two years, envisages checking that said chemico-physical parameters obtained for each oil are comprised in the range of values of the following table:

| Fatty acids in % of the | |
|---|---|
| triglyceride fraction | Range of values |
| C16:0 | 12.0-17.0% |
| C16:1 | 0.4-1.0% |
| C17:0 | 0.05-0.2% |
| C17:1 n-7 | 0.05-0.2% |
| C18:0 | 1.8-3.0% |
| C18:1 n-9 | 68.0-76% |
| C18:2 n-6 | 5.0-10.0% |
| C18:3 n-6 | 0.5-0.9% |
| C20:0 | 0.3-0.5% |
| C20:1 | 0.1-0.4% |
| C22:0 | 0.05-0.2% |
| C24:0 | 0.05-0.2% |
| Σ saturated acids | 15.0-19.0% |
| Σ mono-unsaturated acids | 71.0-77.5% |
| Σ polysaturated acids | 6.0-10.4% |
| Acidity number (% of oleic acid) | 0.2-0.7 |
| Number of peroxides (mEq. O₂/kg) | 6.0-15.0 |
| Total sterols (mg/kg) | 1050-1350 |
| Campesterol (%) | 2.7-3.9 |
| Stigmasterol (%) | 1.5-2.9 |
| β-sitosterol (%) + Δ-5-avenasterol | 89.0-94.0 |
| (%) | |
| cholesterol (%) | 0.1-0.3 |
| others (%) | 0.5-2.0 |
| Total antioxidant power (mmol | 1000-2000 |
| Trolox Equiv./kg) | |
| TEAC method | 1000-2000 |
| RAC method | |
| d-RRR-α-tocopherol (mg/kg) | 200-400 |
| d-RRR-γ-tocopherol (mg/kg) | 3.0-10.0 |
| β-carotene (mg/kg) | 1.0-2.5 |
| Squalene (mg/kg) | 4000-8000 |
| Total phenols (Folin-Chocaltau, mg | 70-200 |
| gallic acid/kg) | |
| Tyrosol (mg/kg) | 7-20 |
| Hydroxytyrosol (mg/kg) | 10-25 |

9. The method according to claim 8, characterized in that it further envisages accompanying each package of oil analysed with a table bearing the values that have emerged from the analyses for each of the items indicated, in order to quote the antioxidant power of the content of each package and recognize the quality of oil to overcome the problem of subjective organoleptic findings.

10. The method according to claim 8, further comprising accompanying each package of oil analyzed with a table bearing the values that have emerged from the analyses for each of the items indicated, and that said values detected are accompanied by the range of values.
